(19) United States
(12) Patent Application Publication
(10) Pub. No.: US 2021/0386581 A1
(43) Pub. Date: Dec. 16, 2021

(54) APPARATUS FOR PORTABLE CONTINUOUS CRYOTHERAPY TREATMENT

(71) Applicants: Casey Michael Mulder, Fort Collins, CO (US); Jason Charles Barta, Loveland, CO (US)

(72) Inventors: Casey Michael Mulder, Fort Collins, CO (US); Jason Charles Barta, Loveland, CO (US)

(21) Appl. No.: 16/898,389

(22) Filed: Jun. 10, 2020

(51) Int. Cl. A61F 7/02

(52) U.S. Cl. CPC A61F 7/02; A61F 2007/0056; A61F 2007/0287

(57) ABSTRACT

A lightweight highly portable scalp or body part cooling system to mitigate the effects of chemotherapy induced alopecia or other medical condition. The device consists of a scalp heat exchanger cap or body part heat exchanger, heat exchange fluid, a DC voltage refrigeration fluid chiller, measurement sensors, battery, voltage converter and a microprocessor packaged in a hard case with wheels and handle for patient portability.

### CROSS REFERENCE TO RELATED APPLICATIONS

This application is based on and claims priority and benefit of Provisional U.S. Patent Application No. 62/859,540 and their entire contents of which are incorporated herein by reference.

### FIELD OF THE INVENTION

This invention relates to people needing cryotherapy treatment while receiving chemotherapy or due to a bodily injury. This invention encompasses a portable cooling system which can be used continuously without the need to exchange the heat transfer material allowing the user to continue cold therapy at the required temperature on the desired body part. More specifically, but not limited to, this invention is best used for the prevention of hair loss while undergoing chemotherapy treatment.

### BACKGROUND AND PRIOR ART

A person undergoing chemotherapy treatment will often experience hair loss as a side effect of the chemotherapy treatment. It has been shown through many studies that by reducing the scalp temperature while the cytotoxic drug is at a hair follicle damaging concentration levels in the blood, hair loss can be greatly reduced or eliminated. It has been found that scalp cooling or cryotherapy can slow the loss of hair or, in some instances eliminating hair loss altogether. Scalp cooling mitigates chemotherapy induced alopecia in 2 ways: 1) As the skin of the scalp cools, the blood vessels contract (vasoconstriction) causing a reduction in blood supply to the hair follicles. This action diminishes the amount of cytotoxic drug in the blood stream that reaches the hair follicles. The result is that the hair follicles will not be as dramatically affected by the cytotoxic drugs and hair loss will be diminished. 2) As the temperature of the hair follicles decrease, the cellular metabolism level decreases at this lower temperature which reduces the amount of chemotherapy drug that is received by the hair follicle cells thereby reducing the damaging effect.

Typically a scalp will be cooled about 20 minutes before active chemotherapy begins, during the active chemotherapy infusion, and then several hours after active infusion has completed to keep the scalp at a reduced temperature until the chemotherapy drug has been sufficiently metabolized by the patient's body to a less damaging level to the hair follicles.

One method of scalp cooling involves applying some form of heat exchanger to the scalp to remove heat and cool the scalp in combination with some form of fluid chiller. Cooling fluid is pumped through a flow passage inside a heat exchanging surface to be applied against the scalp for cooling the scalp.

Currently there are a several solutions for using cryotherapy as a means to reduce hair loss due to chemotherapy. These systems typically consist of a cooling unit with a connected cooling cap and cap tightening system. They are expensive, large, and heavy, limiting their use to inside medical facilities only. Although these systems are the accepted treatment systems in use today for chemotherapy hair loss prevention there are problems and disadvantages to these two systems. These systems consist of a cooling unit with a connected cooling cap and cap tightening system. They are expensive, large, and heavy, limiting its use to inside medical facilities only. Although these systems are the accepted treatment systems in use today for chemotherapy hair loss prevention there are problems and disadvantages to these two systems. (see references section)

Current head heat exchangers caps do not always fit tightly to the head and thus require elaborate, uncomfortable outer caps with various chin strapping that apply inward force to the heat exchange cap. These strapping methods can become uncomfortable after many hours of treatment, make eating and drinking more difficult and can increase patient's anxiety due to the claustrophobic feeling of being attached to and strapped into so many things during infusion.

Current head heat exchanger caps do not provide any cryotherapy to the eyebrow area in their heat exchange cap shape design and thus the eyebrow area is not cooled and most patients will lose their eyebrows during chemotherapy.

Current solutions do not provide any real-time feedback to users of cap fit or scalp temperature. Most solutions provide for 1 or 2 sensors that are integrated into the heat exchange portion of cap but these are used only as an indicator that the heat exchanger cap has not dropped below a safe temperature. To ensure the scalp is cooled to the proper temperature, it would be ideal to measure the scalp temperature in many areas directly with temperature sensors. One issue found in attempting to continually measure scalp temperature with integrated temperature sensors such as thermistors is that because the heat exchange cap is in direct contact with the scalp, it is very difficult to distinguish whether a temperature sensor is actually measuring the scalp temperature or if it is measuring the heat exchange cap temperature. It is theorized that poor or intermittent heat exchange cap to scalp contact and resulting insufficient scalp cooling locally may be the reason for variation in patient success rates of retaining hair during chemotherapy with current solutions.

Current solutions do not allow full patient mobility. Patients must typically remain at their infusion chair during all scalp cooling periods, up to 5-6 hours. While it is possible to disconnect from the chiller unit for a short period of time perhaps to walk to the restroom, scalp temperatures will rise slightly during this period and some degradation in efficacy would be expected.

Current non-mobile solutions require that the patient must remain at the infusion center during the post-infusion chilling period until the level of chemotherapy drug has metabolized to a non-damaging level. This period can be up to several hours. After a long day of the infusion process, patients just want to leave and get back to the comfort of their own home as soon as possible, not remain at an infusion center for hours for post-infusion scalp chilling.

During cryotherapy treatment the patient's body temperature can drop and most patients utilize and external heat source to provide comfort and means maintain body heat body such as pre-warmed blankets or electric blankets. The current solutions do not offer a method to help with this via their machines. The thermodynamic process of using a compressor, evaporator, and condenser must remove energy from the system in the form of heat which can be used to warm the patient during treatment.

Current solutions require the medical staff to be trained on how to use the device, which is expensive and time consuming for the hospital which is then passed on to the patient through higher costs. Current solutions are also large machines which take up valuable space in infusion centers that most do not have.

Current solutions are also expensive for infusion centers to own which limits the number of devices that are typically available to patients at most infusion centers. A typical large city infusion center with 30 infusion bays might treat 20 patients per day that would benefit from scalp cooling. The capital investment for 10 current solution devices might cost a infusion center $500,000-$700,000.

### SUMMARY OF THE INVENTION

Accordingly, the primary object of the present invention is to overcome the deficiencies of the prior art systems by providing a scalp cooling system that for a more shape compliant heat exchange cap that is effective and comfortable for patients. A further need exists for heat exchange cap design to cover the eyebrow area as part of its basic shape. The field also needs a solution that provides real time feedback to patient indicating the heat exchange cap is fitting properly to the head and functioning properly. Finally, there is also a need for a lightweight, portable, fully mobile, completely self-contained continuous cooling solution to improve patients convenience that makes cryotherapy affordable to a much larger demographic and to enables new distribution methods of cryotherapy for more widespread use.

An object of the present invention is to eliminate or alleviate at least one of the drawbacks mentioned above, which is achieved by assigning to the device the characteristics according to the aspects described below.

According to one aspect of the invention, there is provided a head cooler or body part wrap comprising a heat exchange element to be arranged on the head of a patient covering the scalp hair area as well as eyebrow area or body part, and at least one flow passage system for a cooling fluid provided in the heat exchange cap or heat exchange element forming an inside heat exchange surface to be applied against the head or body part for cooling. Very soft durometer materials are used in the construction of the heat exchange cap to provide for a very shape compliant fit thus eliminating the need for additional straps to hold down the heat exchange cap to scalp.

According to a second aspect of the invention, rather than attempting to measure the temperature of scalp directly with temperature sensors, we can infer that if the measured heat exchange fluid is at proper temperature and flow rate, and if the heat exchange cap is in sufficient contact with scalp then the scalp is being cooled to the correct temperature. To ensure the cap is in sufficient contact with scalp, force sensors can be arranged in an array on top of the heat exchange element of the cap to measure the force applied to the heat exchange cap. A force producing cap is fitted on top of the heat exchange cap and force sensors measure applied force across multiple areas over the cap to ensure a uniform even inward force to the heat exchange cap is being provided by the force producing cap thus ensuring good contact between the heat exchange cap and the scalp. Another method of inferring sufficient scalp cooling is the use of heat flux sensors. An array of heat flux sensors is arranged across the scalp and eyebrow area in-between the scalp and heat exchange cap. Heat flux sensors measure the amount of heat transfer between scalp and heat exchange cap thru said heat flux sensor and calculations are made to determine if scalp is at proper cooled temperature.

According to a third aspect of the invention, a thermally insulating element is applied on top of the force producing cap to minimize heat gains of heat exchange cap from ambient air as well as apply the proper pressure to the cap or body part wrap for sufficient cooling.

According to a fourth aspect of the invention, cap force sensors or heat flux sensors and all other operational parameters are monitored by a base unit computer and are wirelessly transmitted to a central location for remote monitoring and analysis. Such data can be analyzed and real-time feedback of cap fit and system performance can be provided to patient via base unit screen or through a personal electronic device such as cell phone or tablet allowing a patient to make any adjustments to the heat exchange cap to rectify any detected issues.

According to a fifth aspect of the invention, a small, DC (direct current), variable speed compressor is used as the chiller component of the system. Being 50% lighter in weight, 50% smaller in size and 30% more efficient, this compact DC variable speed compressor has allowed a novel approach to total system integration to enable a completely self-contained, operational portable system that is unavailable today.

According to a sixth aspect of the invention, the unit can be powered by 110 v, 220 v AC voltage (plugged into a wall outlet) or 12 v on-board battery, or 12 v vehicle power for complete mobility for the patient.

According to a seventh aspect of the invention, the unit can be used to heat the patient's body during treatment. The thermodynamic process that occurs when using a compressor and heat exchangers with refrigerant as the working fluid generates heat which must be dissipated. This heat can be recovered and used via a small blower and tube to transfer the hot air to the patient in order to make them more comfortable during the treatment.

Further objects, features and advantages of the present invention will appear from the following detailed description, from the attached drawings as well as from the dependent claims.

### BRIEF DESCRIPTION OF THE DRAWINGS

The invention will be described in greater detail with reference to the accompanying drawings which represent a preferred embodiment thereof, wherein:

FIG. 1. shows one example of the entire system and the interactions with the cap or body part wrap. The case 101 holds and protects the critical components of the cooling system. The cooling system case has a door 102 to allow for easy serviceability and quick disconnects 105 and 106 for the inlet tube 110 and outlet tube 111. There is a handle 107 and wheels 120 for easy transport and maneuvering over objects. The system has a user interaction touch screen 108 as well as a plug 109 to allow for locating the screen in a convenient location. The inlet tube 110 and the outlet tube 111 are connected to the cap via quick disconnect fittings 112 and 113 and the supply tube 119 is integrated into the cap or body wrap which then is connected through the heat exchanger cap or body wrap to the return tube 114. The cap has three layers; the cap heat exchange element 115, a force sensor matrix sensor 116, and an insulating layer 117.

FIG. 2. shows one example of the internal components of the cooling system in more detail. The system has a chiller system 300 which is described in more detail in FIG. 3, The chiller system required adequate cooling, therefore there are fans 201 including ducting 202 to direct the air to the proper location for efficient chiller operation. There is a pump 210 which is used to transfer fluid through the heat exchanger on the chiller and to transfer fluid to the cap or body wrap as seen in FIG. 1. There is a reservoir 208 where the fluid returns back to from the cap as well as provide some thermal mass for the system. There is a full control system which includes a power supply 204, a compressor controller 205, a 12 volt battery 207, a low voltage shutoff 206, and a system controller 209. All of these components are integrated together with various sensors to fully automate the system.

FIG. 3. shows one example of the details of the chiller system. The chiller system functions by employing vapor compression cycle or Sterling cycle process. In order for the thermodynamic refrigeration cycle process to work a compressor 301, a condenser 302, an evaporator 304 and cooling fans 303 is used.

FIG. 4. shows one example of the force sensing array 116 applied on top or integrated on top of or integrated into heat exchange cap 115 and a force producing element 401 on top of force sensing element 116. Each signal sensor wire channel 403 is connected to the bottom of a series of force sensors and a signal return wire 404 is connected to the top of a series of force sensors. All sensor signal wires and signal return wires are connected to the control unit or multiplexer. The force producing layer 401 provides a uniform inward force 402 to the heat exchange cap 115 to ensure there is sufficient contact between the heat exchange cap and the scalp. Force producing layer can be construct of any elastic material such as Lycra, silicone or latex.

FIG. 5. shows the one example construction method of the force matrix sensors. Many types of pressure sensors could be used in force sensor array such as variable resistive sensors, capacitive sensors, strain sensors or a combination of these sensors. In one embodiment of this invention, a force sensor is comprised of a resistive pressure sensing material 501 sandwiched between a top 502 and bottom conductive element 503. Resistance or voltage drop across the sensor can be transmitted by signal wire 504 and signal return wire 505 As force is applied normally across the conductive layer and transmitted to the resistive layer, a voltage drop can be measured across the conductive layers proportion to the force applied. Many sensors could be connected individually to a control computer to read values of each sensor, but this would require an equally many quantity of sensor wires and equally many sensor inputs in a control device.

FIG. 6. shows one example of how the force sensors can be applied in a matrix in order to properly measure force pressure across the entire cap. Each sensor must be sampled individually. In order to achieve this, the sensors 601 are placed in an array. As can be seen in FIG. 5, the signal wire are shaped as long horizontal rows 602, and the signal return wires 603 are shaped as long vertical columns. Through this sensor/electrode arrangement, and through the use of multiplexers 604, 605 which control the path of current through the electrode rows/columns, each sensor can be isolated and sampled from. In addition, multiplexers have such a high bit rate, that all of the sensors appear as if they are being measured simultaneously.

FIG. 7. shows one example of how the cap is constructed. The heat exchanger cap is comprised of 2 parts: a continuous spiral of two extremely thin wall silicone tubing 701 and a cast silicone molded channel component 702. Heat exchanger element is constructed such that eyebrows are covered 704 in addition to scalp hair area. Gaps 703 are provided in cap construction to allow air to escape that might be trapped between scalp and silicone gap.

FIG. 8. shows one example of a user interface providing cap force feedback to user. When all systems are normal including cap force sensor matrix, a simple normal status is indicated 804. Cap force visualization 806 is shown indicating the status of each section of cap monitored by force sensor matrix. In the event that insufficient force is detected in a section, system status is changed to show abnormality 804, the cap section with abnormality is shown in red 803 for the user to easily understand where the abnormality is, and corrective action needed is shown 805. The system will continually monitor cap force and provide feedback when cap force is back within normal limits.

FIG. 9. shows one embodiment of the system logic flow chart.

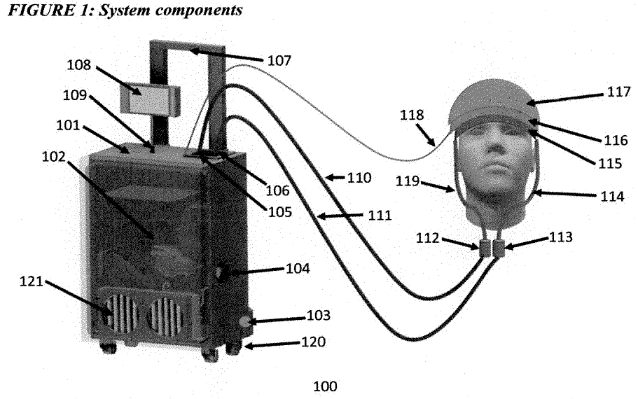

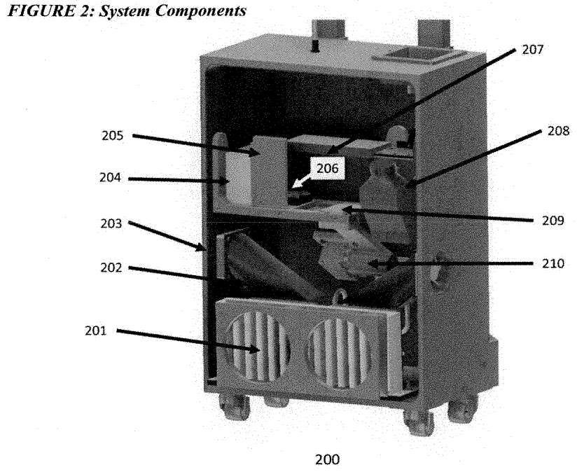

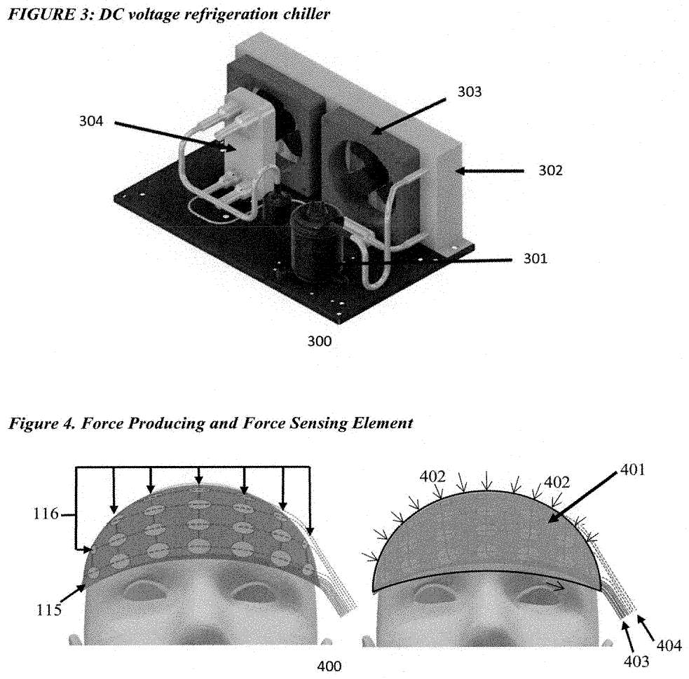

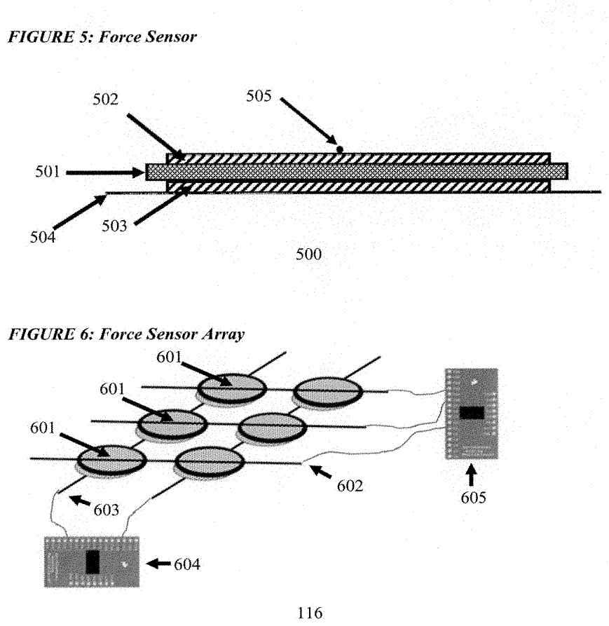

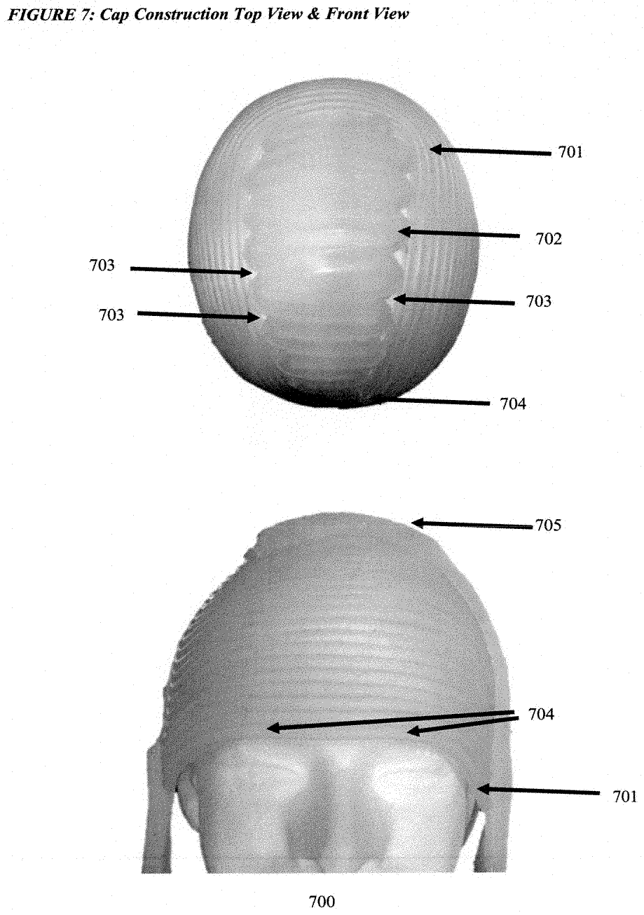

### DETAILED DESCRIPTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

An object of the present invention is to eliminate or alleviate at least one of the drawbacks discussed in the background, which is achieved by assigning to the device the characteristics according to the description below.

According to one aspect of the invention, there is provided a head cooler or body part wrap comprising a heat exchange method to be arranged on the head of a patient covering the scalp hair area as well as eyebrow area or body part, and at least one flow passage system for a cooling fluid provided in the heat exchange cap forming an inside heat exchange surface to be applied against the head or body part for cooling. Very soft durometer silicone materials are used in the construction of the heat exchange cap to provide for a head shape compliant fit thus eliminating the need for additional straps to hold down the heat exchange cap to scalp to achieve good heat exchanger to scalp contact and heat transfer. Silicone tubing is used to construct the lower portion of the heat exchanger cap by wrapping tubes circumferentially a head shape generally following the lower scalp hair line and eyebrows 704 upwards towards the apex of a head shape. For best thermal transfer a silicone tube wall thickness of 0.5 mm is used and for optimal conformity to scalp a soft durometer Shore 50 tubing is used. To ensure heat exchange fluid does not increase in temperature too much to impede heat transfer as it flows around the flow path from bottom of hairline to top, two tubes parallel to each other are used to reduce the flow path by half and reduce the amount of heat that would be absorbed in just one tube. Because of the tubing thin wall and soft durometer, this tubing can only be curved to a radius of approximately 20 mm before the tubing will kink. Therefore, silicone tubing is used for approximately 24 rows to the point where the curve radius is too small to continue. To construct the remaining heat exchanger cap, a cast silicone section 702 is made with a single effective flow area the same as the two silicone tubes 701 combined in the lower construction of the heat exchange cap. The cast silicone component can be fabricated with a soft durometer silicone for the section that will be in contact with the scalp and a higher durometer silicone for the outer section to provide strength. A series of small holes 703 can be cast into the silicone cast component to allow for additional sensors to be inserted or to allow air to escape between the scalp and cap for a tighter fit.

The silicone tubing section of the heat exchange cap is bonded with silicone peripherally to the silicone cast component. The two silicone tubes are inserted into the silicon cast component and bonded with silicone. A single return tubing line 705 is inserted into the outlet of the silicone cast component is bonded to the cast silicone component. Because of the very soft durometer silicone and extremely thin tube wall thickness used for most of the construction of the heat exchanger cap, it is shape compliant to patients head and requires just a small amount of uniform inward force to effectively provide good heat exchanger cap to scalp contact. A force producing element or force producing cap constructed out of elastic stretch material such as Lycra or silicone is applied on top of heat exchange cap to provide slight uniform inward force to the heat exchange cap to ensure good contact between the heat exchange cap and the scalp. Other solutions have consistently used a higher shore durometer (harder) silicone for ease of manufacturing, but this has required them to employ more complex and uncomfortable strapping methods to attempt to get good scalp to heat exchanger contact. This novel approach of using very low durometer silicone tubing in combination with a low durometer silicone cast component for the heat exchanger element provides a much more compliant fit for better heat exchange while eliminating fabrication issues with softer durometer materials. Additionally, the present invention heat exchange cap fully encompasses not just hair area of scalp but also eyebrow areas reduce hair loss to the eyebrows.

According to a second aspect of the invention, to ensure the scalp is cooled to the proper temperature, it would be ideal to measure the scalp temperature in many areas directly with temperature sensors. Heat exchange caps are in direct contact with the scalp and therefore it is very difficult to distinguish whether a temperature sensor is actually measuring the scalp temperature or if it is measuring the heat exchange cap temperature. Rather than attempting to measure temperature of scalp directly with temperature sensors, we can infer the scalp temperatures by first measuring the heat exchange fluid temperature and flow at the control unit and then measuring the force applied to the heat exchange cap. To ensure the cap is in sufficient contact with scalp, force sensors can be arranged in an array on top of the heat exchange element of the cap to measure the force of the force producing cap to the heat exchange cap. If the heat exchange fluid supply is at the proper temperature and flow rate, and the heat exchange cap force sensors measure sufficient force, the control unit can infer proper cooling is occurring uniformly across the scalp. With this real-time data of scalp to heat exchanger cap force and heat exchange fluid temperature and flow, the unit can provide much needed assurance to the patient that not only is the control unit and chiller functioning properly but also the cap is functioning properly and the patient can worry about one less thing during infusion. This novel way of inferring proper scalp temperature by measure heat exchanger cap force and heat transfer fluid temperature and flow allows a significant improvement in monitoring and system performance effectiveness compared to current solution.

Another method of inferring sufficient scalp cooling is the use of heat flux sensors. Heat flux sensors measure the amount of heat transfer between scalp and heat exchange cap thru said heat flux sensor and calculations are made to determine if scalp is at proper cooled temperature. An array of heat flux sensors is arranged across the scalp and eyebrow area in-between the scalp and heat exchange cap. If the heat exchanger cap is not in sufficient contact with the scalp, the measured heat flow through a heat flux sensor will be lower than expected and the control unit can provide user feedback of the local area deficiency.

According to a third aspect of the invention, a thermally insulating element cap is applied on top of the force producing cap to minimize heat gains of heat exchange cap from ambient air as well as apply some additional inward force to the cap or body wrap for sufficient cooling. In one embodiment, Aerogel insulation panels are incorporated into the thermally insulating cap to provide 3 times the insulating value compared to neoprene type insulation typically used in other solutions.

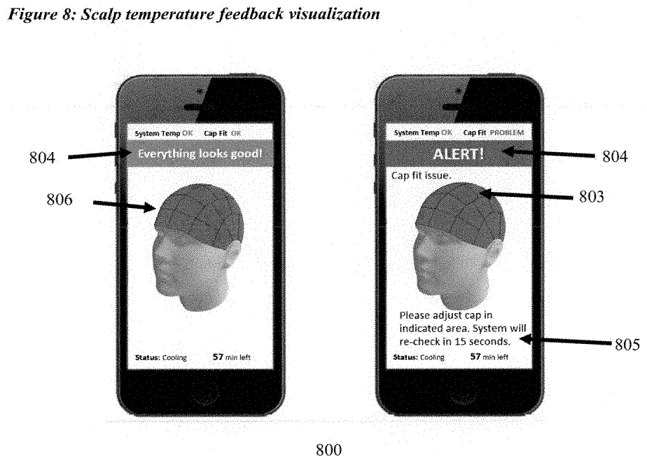

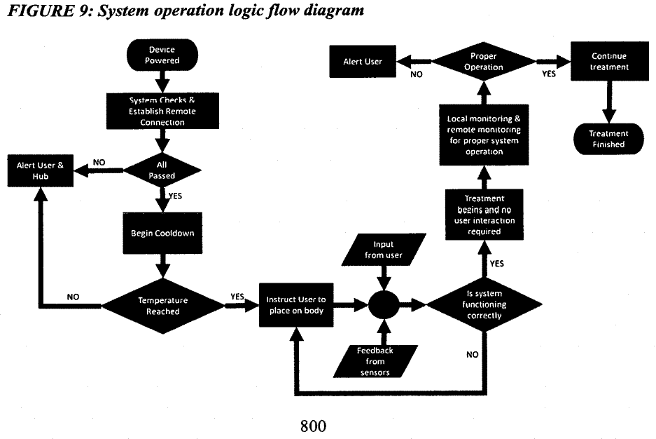

According to a fourth aspect of the invention, cap force sensors or heat flux sensors and all other operational parameters are monitored by base unit computer and are wirelessly transmitted to a central location for remote monitoring. Such data can be analyzed, and real-time feedback of cap fit and system performance can be provided to patient via base unit screen or thru a personal electronic device such as cell phone or tablet. A key aspect of successful cryotherapy to reduce hair loss during chemotherapy is ensuring all key engineering parameters are being met. Medical facilities staff are typically not trained well in understanding critical engineering factors in cryotherapy treatment and having a trained person onsite to continually monitor every patient undergoing cryotherapy is impractical. Easy to understand real-time feedback to the user is essential to successful cryotherapy. Because cap fit and force measurements are one of the most critical aspects of treatment effectiveness, an easy to understand visualization of cap fit and force is provided to the user via unit display screen or other connected device screen as shown in FIG. 8. The control unit continually monitors the array of force sensors with a sampling rate of approximately 1 Hz. The control unit will average force measurements over time and if a sustained period of insufficient force is detected a user alert will trigger. The user can visually see on the display the area of concern and take corrective action to better seat the heat exchange cap to scalp. The control unit will change the alert status to normal if it calculates a nominal value over a sustained period of time. All other key operational parameters of interest to the patient will be shown along with any detected faults with the system along with any corrective action instructions. Our visually rich interface to the patient will provide them real time relevant information that they can understand and take action upon that current solutions do not provide.

According to a fifth aspect of the invention, the unit can be powered by 110 v, 220 v AC voltage (plugged into a wall outlet) or 12 v on-board battery, or 12 v vehicle power for complete mobility for the patient. Power supply is automatically detected and switched as the user changes power supply sources throughout their cryotherapy treatment and location. (infusion center, walking to bathroom, walking to car after infusion, in the car on the way home) The on-board battery supply will provide sufficient power for at least 35 minutes of treatment to allow patient to walk from infusion to car where they could then plug into vehicle 12 v power supply. Battery level is monitored and system alerts will inform user of low power to instruct them to plug into another power source within a prescribed amount of time. This multi-voltage capability and battery power storage approach is one of the key aspects that allows the patient to be fully mobile in and out of the medical facility, walking, and in their car providing a substantial relief of some of the burden patients bear with current solutions.

According to a sixth aspect of the invention, a small DC variable speed compressor is used in a vapor-compression refrigeration cycle in the chiller component of the system. In a further embodiment of this invention, a DC variable speed compressor is used in a Sterling Cycle refrigeration cycle in the chiller component of the system. Traditional compressors running on 110 v AC supply are typically at least 3 times as big and 10 lbs heavier compared to this DC variable speed compressor. DC variable speed compressors are typically 30% more energy efficient compared to 110 v AC compressors. This 30% higher efficiency allows for a smaller battery to be used in this system compared to a battery and inverter needed to power a 110 v AC compressor. A smaller and lighter compressor using a smaller and lighter battery both contribute to a much smaller and lighter total system which is key to the mobility and portability to this solution. Due to this compact design and lightweight design, this device can be shipped directly to the patient's home by standard delivery services (UPS, Fed-ex) and offers a paradigm shift for the convenience of patients cryotherapy experience at a much lower cost than traditional methods.

According to a seventh aspect of the invention, the unit can be used to heat the patients body during treatment. During cryotherapy heat is removed from the scalp or body part of patient. This heat loss can lead to patient discomfort and shivering. Patients typically use warmed blankets; hot water bottles or electric heating pads to stay warm. The thermodynamic process that occurs when using a compressor and heat exchangers with refrigerant as the working fluid generates heat which must be dissipated. This heat can be recovered and used via a small blower and tube to transfer the warm air to the patient in order to make them more comfortable during the treatment.

We claim:

1. A portable cryotherapy system to cool down and maintain a desired temperature of a person's body part that is comprised of:
a) a flexible and conformal heat exchanger with at least one contiguous fluid conduit from an inlet connector to an outlet connector formed to substantially encapsulate the body part to be cooled,
b) a hard body case affixed with wheels and telescoping handle for self-patient portability
c) a control system and patient interface that provides feedback and instructions to patient as well as data transmission to remote database.
d) a thermally insulated fluid tank and plumbing cooler system with a tank, and variable speed DC compressor in a vapor-compression refrigeration cycle or Sterling refrigeration cycle, a positive displacement pump to circulate the transfer fluid, electrical components to control and power the system including an uninterruptible power controller to automatically switch between battery power and external power sources.

2. The portable cryotherapy system according to claim 1 wherein a heat exchanger covers all of patient's hair scalp area as well as eyebrow area or body part,
wherein at two cooling fluid flow passages are constructed with thin walled soft durometer silicone tubing and are constructed by circumferentially wrapping tubing around a head shape generally following the lower scalp hair line and eyebrows upwards towards the apex of a head shape.
wherein lower heat exchanger section of silicone tubing is constructed with a series of gaps between silicone tubing rows to allow for trapped air between scalp and heat exchange cap to escape
wherein a molded cast silicone fluid flow passage piece is provided in the top section of the heat exchanger construction for the apex of head
wherein the silicone tubing section of the heat exchange cap is bonded with silicone peripherally to the silicone cast component.
wherein a force producing element or force producing cap constructed out of elastic stretch material such as Lycra or silicone is partially affixed to heat exchange cap

3. The portable cryotherapy system according to claim 1 wherein the heat exchange cap further comprises an array of force pressure sensors.

4. The portable cryotherapy system according to claim 1 wherein the heat exchanger cap further comprises of at least 2 heat flux sensors where sensor data is used to infer proper cap and heat transfer.

5. The scalp cooling system according to claim 1 wherein force pressure sensor signals are used in conjunction with other sensors to infer proper cooling and fitment.

6. The portable cryotherapy system according to claim 1 wherein the system can be powered by AC electrical power, and/or DC electrical power and automatically switch power sources when either source is connected or disconnected.

7. The portable cryotherapy system according to claim 1 wherein the system incorporates a battery energy storage that allows system to be powered solely by onboard battery.

8. The portable cryotherapy system according to claim 1 wherein a small DC variable speed compressor is used in a refrigeration cycle in the fluid chiller component of the system.

9. The portable cryotherapy system according to claim 1 wherein the waste heat generated from refrigeration cycle to is provided back to patient for body warming.
